(19) United States
(12) Patent Application Publication
(10) Pub. No.: US 2024/0050498 A1
(43) Pub. Date: Feb. 15, 2024

(54) SYNERGISTIC COMPOSITION OF PHAGES AND ITS FORMATION METHOD

(71) Applicant: Proteon Pharmaceuticals S.A., Lódz (PL)

(72) Inventors: Jaroslaw DASTYCH, Lódz (PL); Ewelina A. WÓJCIK, Grabica (PL); Agnieszka MASZEWSKA, Lódz (PL); Malgorzata STANCZYK, Lódz (PL); Anna PEKALA, Lódz (PL); Edyta SMIGIELSKA, Lódz (PL); Ewelina WOJDA, Lódz (PL); Joanna KAZIMIERCZAK, Lódz (PL); Arkadiusz GUZI?SKI, Lódz (PL); Justyna ANDRYSIAK, Lódz (PL); Paulina WIGNER, Lódz (PL)

(73) Assignee: Proteon Pharmaceuticals S.A., Lódz (PL)

(21) Appl. No.: 18/012,318

(22) PCT Filed: Jun. 28, 2021
(86) PCT No.: PCT/PL2021/050047

§ 371 Date: Dec. 22, 2022

(30) Foreign Application Priority Data
Jun. 26, 2020 (PL) P.434485

(51) Int. Cl. A61K 35/76; C12N 7/00; A61P 31/04

(52) U.S. Cl. CPC A61K 35/76; C12N 7/00; A61P 31/04

(57) ABSTRACT

The formation method was revealed of a bacteriophage synergistic composition intended to prevent and combat infections of livestock, including but not limited to poultry, with pathogenic bacteria strains sensitive to the phages.

### RELATED APPLICATIONS

This application is a National Phase of PCT Patent Application No. PCT/PL2021/050047 having International filing date of Jun. 28, 2021, which claims the benefit of priority of Poland Patent Application No. P.434485 filed on Jun. 26, 2020. The contents of the above applications are all incorporated by reference as if fully set forth herein in their entirety.

### FIELD AND BACKGROUND OF THE INVENTION

The invention relates to the formation method of synergistic compositions of phage strains specific against a selected bacteria strain and a synergistic composition of phages obtained with this method, and the application of adequately composed bacteriophages to produce a synergistic preparation composition to prevent and eliminate infections of livestock, including but not limited to poultry, with pathogenic bacteria strains sensitive to the phages.

Poultry meat production has been the fastest developing segment of the global meat market for many years now. According to the USDA data, global production of chickens amounted to 98.4 min tons in 2019, which means 3% more than in 2018. What is more, according to the OECD-FAO report, global meat consumption will reach the level of 131 min tons by 2025 [Short-term outlook for EU, 2019].

Bacterial diseases are among the leading causes of economic losses in poultry production. Avian pathogenic Escherichia coli strains (APEC) are significant etiological factors of bird bacterial diseases. They cause colibacillosis—one of the most infectious poultry diseases [Mellata M., 2013].

Many years of using antibacterial drugs, including but not limited to antibiotics in the poultry sector, significantly contributed to the development of drug resistance in different bacteria strains, such as APEC [Dho-Moulin et al., 1999]. The resistant strains, which can be transmitted by the infected poultry, pose a problem for selecting effective antibiotic therapy for animals and humans A continuous increase in the number of colibacillosis cases, limitations imposed by the EU in relation to the use of antibiotics and high drug resistance of E. coli strains force the need to look for an alternative and effective method to combat the pathogens in the poultry sector, which will not only help reduce significant economic losses caused by high mortality of the poultry but also cut down on the cost of treatment.

One of the proposed methods to eliminate the infections caused by pathogenic bacteria is the use bacteriophages (phages) as natural viruses which are able to destroy specific bacteria groups. Phage therapy is mentioned most commonly among the alternative, effective and safe biological method to combat infections with antibiotic-resistant pathogens [Furfaro L. L. et al., 2018]. The results of in vivo tests confirm high efficacy and safety of phage preparations used in veterinary medicine, also to protect poultry from colibacillosis. The adequate selection of the bacteriophage cocktail ingredients and the selection of the preparation application method are the significant factors which affect the efficacy of phage therapy. Huff et al. revealed that endotracheal administration of phages immediately after pathogen infection resulted in reduced mortality among the chickens infected with APEC strains [Huff W. E. et al., 2013]. The administration of phages to birds in spray to prevent infection with E. coli reduced the chickens' mortality (7%) compared to the control group (63%) Simultaneous administration of phages with a mixture of pathogenic bacteria into the air sacs helped to reduce the mortality to 5% in the group which received bacteriophages, whereas in the control group it amounted to 85%. Intramuscular injection of bacteriophages protected the birds against severe E. coli infection and reduced the mortality to ca. 20% [Huff W. E. et al., 2005]. Interestingly, in vitro studies revealed that ca. 70% of nearly 150 studied APEC strains were sensitive to the mixture of phiF78E, phiF258E and phiF61E phages, whereas only slightly more than 50% of the bacteria strains were sensitive to the applied antibiotic (enrofloxacin) [Oliviera A. et al., 2009]. Intramuscular injection of SPR02 or DAF6 phages reduced the chickens' mortality up to 10% [Huff W. E. et al., 2006]. High activity against O1, O2 and O78 E. coli serotypes characterised EC-Nidi and EC-Nid2 bacteriophages, causing a decrease in the birds' mortality [Jamalludeen N. et al., 2009]. Endotracheal administration of the phages in chickens with colibacillosis resulted in a significant decrease in the treated animals' mortality to 13% compared to 83% in the non-treated group. Furthermore, phage therapy prevented the augmentation of the liver, pancreas and the heart [Lau G. L. et al., 2010]. On the other hand, Huff et al. (2002) did not observe any impact of phage therapy on the studied organs' size. Moreover, a slowdown or inhibition of the general growth was observed in the poultry infected with pathogenic E. coli strains [Dunnington E. A. et al., 1991; Gomis M. S. et al., 1997], whereas the therapy with EC1 bacteriophage not only reduced the infection severity but also contributed to an improvement in the chickens' growth [Lau G. L. et al., 2010; Naghizadeh M. et al., 2019]. Studies carried out by Huff et al. (2003a, b) did not confirm the influence of phage-containing spray on the body mass index, but also revealed a higher efficacy of phage therapy in poultry, once it was used on the early stage of the disease development [Huff W. E. et al., 2003a]. The F78E phage administration per os or in the form of spray used in chicken houses reduced the chicken's mortality and morbidity following experimental infections with APEC by ca. 25% and 42%, respectively [Oliviera A. et al., 2010]. Similarly, the application of phage preparations onto the litter helped to increase the body weight and decrease the mortality of chickens with colibacillosis caused by direct exposure to pathogens present in farming litter [El-Gohary F. A. et al., 2014]. The application of phage spray preparations onto the animal's beak or as a drinking water additive did not confirm their efficacy in colibacillosis treatment in poultry [Huff W. E. et al., 2002; 2013]. On the other hand, exposing the chickens to a phage preparation mist in a chamber for 3 minutes or endotracheal administration of the preparation prevented the onset of colibacillosis [Huff W. E. et al., 2002a; b; 2013]. Another preparation dedicated for chickens and pigs to prevent opportunistic infections contained a cocktail of at least two phages, specific towards extraintestinal pathogenic Escherichia coli (ExPEC) and APEC. The proposed preparation can be used as a powder, administered as a feed additive or drinking water additive [WO2019051603A1]. The use of φWAO78-1 bacteriophage, specific towards E. coli O78 serotype, in the form of feed additive, can be effective in the treatment and prevention of colibacillosis [KR101275263B1]. Similarly, ΦCJ18, ΦCJ23, ΦCJ24 or ΦCJ25 phages used individually for preventive and therapeutic purposes as a feed additive or drinking water additive or in the form of a disinfectant, can effectively reduce poultry mortality [EP3133151A1; US20160083695A1; WO2015160164A1; KR101299179B1]. The preventive and therapeutic action was also confirmed for EC13 phage applied in the form of a feed additive and drinking water additive [KR101830900B1]. The composition based on EC14 phage in APEC and ETEC infections is intended for use as a feed additive and disinfectant but can also be used to prepare cleaning preparations [KR101871338B1]. Preparation in the form of feed additive and disinfectant for use in the prophylaxis and therapy directed to prevention of infections caused by E. coli O78 serotype in poultry was also developed [KR20120111535A]. Studies carried out in chickens with sepsis, encephalitis, or meningitis confirmed the efficacy of intramuscular injection of R phage injection in the prevention and treatment of E. coli infections [Barrow P. et al., 1998].

The results of studies suggest that the method of bacteriophage administration has a significant impact on phage therapy efficacy. The attempt failed to develop a method of bacteriophage administration to chickens suffering from colibacillosis, optimum for the poultry sector [Huff W. E. et al., 2013]. Decreasing the same bacteriophage's therapeutic efficacy in another therapy poses a certain limitation for phage therapy. Studies carried out by Huff et al. (2010) confirmed that an acquired immunological response (increase in the titer of IgG antibodies) might develop in the poultry, which causes a decrease in the efficacy of the therapy with the previously used phage by as much as 40% [Huff W. E. et al., 2010].

The actual practical problem is the bactericidal suitability of the applied bacteriophage preparations, resulting from the evolution of bacterial strains subjected to the preparation action. It applies primarily to veterinary bacteriophage preparations used for antibacterial protection of meat production in intensive farms, including but not limited to protecting farms producing poultry and/or eggs from E. coli infection. In such places, due to the number of animals and production intensity, the risk of the appearance of bacterial strains resistant to the bacteriophage preparation during its use is relatively high and increases with the time of the same bacteriophage preparation use.

The purpose of the invention is to provide a bacteriophage preparation with extended bactericidal ability. The particular purpose of the invention is to provide a bactericidal composition with extended bactericidal ability, suitable for effective long-term combatting of bacteria pathogenic for poultry, including but not limited to APEC, whose occurrence is particularly adverse in intensive poultry farming.

### SUMMARY OF THE INVENTION

Unexpectedly, the solution to the presented problems is provided in this invention. The invention enables obtaining bacteriophage compositions with a synergistic action, i.e. a composition which contains at least two phage strains with a complementary action. In the reference invention context, the synergistic phage composition stands for a composition of at least two phage strains which combined bactericidal action observed towards the same bacterial strain is higher than the bactericidal action of each strain observed separately towards the bacterial strain.

The authors' discovery of the occurrence of a synergistic action of different phage strains lies at the invention's base. In the course of the research, the authors of the invention observed that for some bacteriophage strains their combined bactericidal action towards the same bacterial strain is significantly higher than the bactericidal action of each strain separately towards the same bacterial strain. Furthermore, it was unexpectedly discovered that synergistic phage compositions have an extended bactericidal ability compared to phages or phage compositions which are not synergistic compositions. A thorough explanation of the mechanism of this phenomenon requires further research. The authors suspect that the phages that form the synergistic phage composition (hereinafter referred to as the synergistic cocktails) combat the same bacterial strain differently. The mechanism of the bacterial strain becoming resistant to the phage's action requires perpetuating the change in the bacterial strain genome to enable effective disturbance in the phage action. In different phage strains with similar molecular action, introducing a change that effectively interferes in the mechanism, leads to the loss in the phage strains' bactericidal activity. Such an effect can be avoided if the phage strains combatting the same bacterial strain use significantly different action mechanisms. Consequently, reaching the resistance to the action of synergistic phage compositions requires a simultaneous introduction of different changes in the combatted bacteria strain genome, which apply to separate mechanisms related to the action of the phages in the synergistic composition. Not only the requirement of the simultaneous introduction of the changes in the combatted bacterial strain genome but also the necessity to modify two separate elements of a bacterial cell are the probable constraints preventing the effective perpetuation of such changes, which can often lead to severe dysfunction of the cell. Eventually, the synergistic phage composition is not only more active, but its bactericidal ability is also much longer.

The subject of the invention includes a process for preparing a synergistic phage composition characterised in that:
a) at least two different phage strains are obtained, specific towards the same bacterial strain,
b) bactericidal activity of each phage separately and their mixture is determined towards this bacterial strain,
c) bactericidal activity determined for each phage used separately is compared with the bactericidal activity determined for their mixture, and
d) if the bactericidal activity of a mixture is discovered to be higher than the bactericidal activity of each phage separately, the phages are considered to form a synergistic composition.

Preferably, on stage b) in order to determine the bactericidal activity of phages, bacterial culture is cultivated in the presence of examined phages, and a bacterial strain is cultivated without the phages, the growth curves are plotted for each culture and the areas under the curves are determined, whereby the ratio of the area under the growth curve determined for the culture without the phages to the area under the growth curve defined for the culture in the presence of the examined phages is regarded as the measure of the examined phages bactericidal activity, and preferably the value of the coefficient N standing for the ratio is determined.

Preferably, on stage b) the growth curves are determined, by measuring at the wavelength λ=620 nm of the samples collected from the tested bacteria strain culture.

Preferably, on staged) the value of coefficient S is determined, being the ratio of the mixture's bactericidal activity to the bactericidal activity of the phage strain with the strongest action in the mixture, whereby the mixture is regarded as a synergistic phage composition if the value of coefficient S is greater than 1.

Preferably, in stage a) in order to obtain a phage strain specific towards the selected bacteria strain:
a collection of bacteriophage strains is obtained, containing a bacteriophage strain specific towards the selected bacterial strain,
a selected bacterial strain is cultivated on a sterile culture medium,
culture samples of individual bacterial strains suspended in the agar are poured onto a solid culture medium, after the top agar has solidified, the suspension of the tested bacteriophage strain with the titer of at least 1×10⁶ PFU/ml is spotted and incubated at 37° C. for at least 18 h,
after incubation, the clear zones are checked in the area where the given bacteriophage strain suspension was applied, which means the inhibition of the bacteria growth. For a bacteriophage strain specific against a selected bacterial strain, clear zones are observed within the top agar,
genetic analysis of the selected phages is carried out with the RAPD or RFLP method and sequencing with the NGS method, which enables the exclusion of the phages which use a lysogenic cycle, and possibly
the identified bacteriophage strain specific for bacterial strains of different serotypes is propagated.

Preferably, APEC strain is the selected bacterial strain.

Another subject of the invention is a synergistic composition of phages specific towards APEC bacteria, containing at least two bacteriophage strains selected from the strains deposited in the Polish Collection of Microorganisms at deposit numbers: F/00137 (215Ecol030PP strain), F/00138 (216Ecol046PP strain), F/00139 (232Ecol030PP strain), F/00140 (235Ecol030PP strain), and F/00141 (236Ecol005PP strain). Preferably, the synergistic composition of phages according to the invention contains at least three of the indicated bacteriophage strains, more preferably it contains at least four of the indicated bacteriophage strains, and most preferably it contains the five indicated bacteriophage strains.

Another subject of the invention is a composition according to the invention, as defined above, for use in the treatment or prevention of livestock infections, including but not limited to poultry, with APEC strains.

Preferably, the composition for use according to the invention is to be administered to the threatened animals as a drinking water additive or spray.

Preferably, the composition for use according to the invention ensures a significant reduction in the mortality of poultry infected with APEC strain.

Preferably, the composition for use according to the invention ensures a significant improvement in the zootechnical parameters (BW, ADFI, ADG, ADWI, FCR and EPEF) in poultry infected with APEC strain.

Preferably, the composition for use according to the invention remains stable for at least 8 months at 4° C. and for at least 8 weeks at 40° C.

The subject of the invention also included a bacteriophage strain suitable for preventing or combatting infections with APEC strains, selected from a group consisting of: F/00137 (215Ecol030PP strain), F/00138 (216Ecol046PP strain), F/00139 (232Ecol030PP strain), F/00140 (235Ecol030PP strain), and F/00141 (236Ecol005PP strain).

Specific embodiment of the invention includes the use of phages (215Ecol030PP, 216Ecol046PP, 232Ecol030PP, 235Ecol030PP, and 236Ecol005PP) for forming a synergistic composition of BAFACOL bacteriophage preparation to prevent and combat bacterial infections in poultry farms, including but not limited to infections caused by APEC strains, whereby preferably the preparation is intended for administration to the threatened animals as a drinking water additive or spray.

In the preferable embodiment of the invention, the composition of BAFACOL bacteriophage preparation specific towards a wide range of poultry pathogenic bacteria strains, preferably APEC, is the synergistic phage composition.

In the preferable embodiment, the preparation composition is obtained with a method, during which:
a. a collection of various poultry pathogenic strains, preferably APEC, is obtained;
b. a collection of bacteriophage strains is obtained using the acquired collection of poultry pathogenic strains, preferably APEC;
c. the bacteriophages specificity towards the poultry pathogenic strains, preferably APEC, is determined as the first stage of selecting the ingredients of a composition with a synergistic action;
d. the lytic activity of bacteriophages is tested, using the spectrometric measurement of the density of the bacteria culture density treated with bacteriophages in time, at the wavelength λ=620 nm, as the second stage of selecting the ingredients of a synergistic composition;
e. initial molecular differentiation of the bacteriophages is carried out, based on the profiles identified with the following methods: Restriction Fragments Length Polymorphism (RFLP) or Random Amplification of Polymorphic DNA (RAPD) as the third stage of selecting the ingredients of a synergistic composition;
f. an analysis of the bacteriophage genome sequencing is carried out, enabling the exclusion of the phages performing a lysogenic cycle from the collection;
g. the activity spectrum of the bacteriophages is determined towards the poultry pathogenic strains, preferably APEC, with diversified surface antigens, defined based on APEC serotypes which are the most common cause of poultry infections; the serotypes are determined in silico, based on bioinformatic analysis of the bacterial genomes;
h. bacteriophages exhibiting only a lytic cycle are selected for a bacteriophage preparation with a potential synergistic action; the bacteriophages are characterised by a wide and/or complementary range of hosts among the poultry pathogenic strains, preferably APEC, with special consideration for serotypes being the most common cause of poultry infections, which helps to eliminate the highest possible number of different poultry pathogenic strains, preferably APEC, obtaining a synergistic action and stronger inhibition of the selected APEC strains' growth, and reducing the probability of the bacteria becoming resistant to the bacteriophages included in the preparation;
i. for a bacteriophage preparation meant to prevent and combat bacterial infections in poultry farms, including but not limited to infections caused by APEC strains, the coefficient S is determined, which describes the synergistic effect of the action of bacteriophages in the preparation towards selected poultry pathogenic strains, preferably APEC.

The revealed method is used in selecting the composition of BAFACOL bacteriophage preparation, demonstrating a synergistic effect, and offering a broad specificity spectrum towards the poultry pathogenic bacteria, preferably APEC, which is significant for industrial applications.

Preferably, the produced bacteriophage composition demonstrates a strong protective and therapeutic action, because it reduces the mortality of poultry experimentally infected with the parenteral E. coli strain pathogenic for poultry.

Preferably, the combatted infection is the infection with poultry pathogenic bacteria, including but not limited to APEC strains, while bacteriophage strains revealed in this application and deposited on 14 Nov. 2019 in the Polish Collection of Microorganisms at the following deposit numbers F/00137 (215Ecol030PP strain), F/00138 (216Ecol046PP strain), F/00139 (232Ecol030PP strain), F/00140 (235Ecol030PP strain), and F/00141 (236Ecol005PP strain) are used for making the preparation.

The special embodiment of the invention covers bacteriophage strains 215Ecol030PP, 216Ecol046PP, 232Ecol030PP, 235Ecol030PP and 236Ecol005PP, which combination provides a synergistic effect aimed to prevent or combat poultry infections caused by bacterial pathogens, preferably with APEC strains.

BAFACOL bacteriophage preparation, according to the invention, is based on the ecosystem's natural ingredients and does not have a negative impact on organisms other than specific pathogenic bacteria.

BAFACOL bacteriophage preparation guarantees that only APEC strains are selectively limited.

BAFACOL bacteriophage preparation is suitable for use in animal production, especially to combat bacterial pathogens, preferably APEC strains, in poultry farms.

The bacteriophage strains revealed in this application were identified owing to the method according to the invention. It was unexpectedly revealed that a mixture of 215Ecol030PP, 216Ecol046PP, 232Ecol030PP, 235Ecol030PP and 236Ecol005PP bacteriophages has a synergistic activity, which provides a broad specificity of the preparation, which involves lysis of at least 82 different APEC strains. The revealed bacteriophage strains remain stable for at least 8 months at 4° C. (min. 90% activity maintained) and for 8 weeks at 40° C. (min. 70% activity maintained).

### BRIEF DESCRIPTION OF THE SEVERAL VIEWS OF THE DRAWINGS

For a more precise explanation, the invention is illustrated in the enclosed figures, where:

FIG. 1 shows the results of comparative studies on the action of bacteriophages used individually or in a cocktail on a selected bacterial strain, which enabled the determination of a synergistic or antagonistic effect of the phages used in the cocktail

FIG. 2 shows the morphology of the bacteriophages used in BAFACOL bacteriophage preparation.

FIG. 3 shows the influence of 215Ecol030PP bacteriophage on the growth of selected bacterial strains.

FIG. 4 shows the results of BAFACOL preparation effectiveness tests when the preparation contains equal amounts of five bacteriophages, disproportionate amounts of the same five bacteriophages, and equal amounts of four out of five selected bacteriophages.

FIG. 5 shows BAFACOL preparation temperature stability tests results when the preparation was stored at 40° C. for 8 weeks and at 4° C. for 8 months.

This description was completed with the following examples aimed to better illustrate the reference invention. The examples shall not be regarded as a full scope of the invention.

### DESCRIPTION OF SPECIFIC EMBODIMENTS OF THE INVENTION

### Example 1. Isolation of bacteriophages Which Ensure the Synergistic Effect of BAFACOL Preparation Composition

Isolation of Bacteriophages Active Towards Selected APEC Strains from Environmental Samples

Eighty-six different strains of Escherichia coli, isolated from poultry and identified with the method described in the patent application P. 431942 as APEC, were used to isolate the bacteriophages. The collection is the property of Proteon Pharmaceuticals S.A.

The isolation of bacteriophages was performed using the following methods: inoculating the bacterial suspension together with the environmental sample in double-layer agar and enrichment protocol of phage particles. Samples of wastewater used as the environmental material for searching the bacteriophages were collected from the inlet collection pipe from the Wastewater Treatment Plant in Łódź, Municipal and Housing Services Department in Strykow, and samples from poultry farms.

One hundred twenty-three (123) bacteriophages active towards APEC strains were selected and subjected to at least 5-times passaging (subculturing) of a single plaque among the plaques obtained on a solid medium to obtain pure bacteriophage strains. In order to select potential ingredients of BAFACOL preparation composition, revealing a synergistic effect, the isolated bacteriophages were characterised, involving: studying the phages specificity towards APEC strains from Proteon Pharmaceuticals S.A. collection, evaluation of the phages lytic activity, bacteriophages differentiation with the RFLP or RAPD method, bioinformatic analysis of the phages genome sequence to determine their similarity, taxonomy and nature (virulent or temperate) and analysis of the phages specificity towards different APEC serotypes. Finally, the coefficient S of the preparations was calculated, indicating the optimum composition of a synergistic preparation. The analyses above allowed to select the phages with the adequate ratio of their counts, ensuring a synergistic effect of BAFACOL bacteriophage preparation composition.

### Testing Bacteriophage Specificity (Host Range)

The specificity spectrum (host range) towards APEC strains was determined for the isolated bacteriophages with a spot test method. For this purpose top agar (medium with a lower agar content) with the addition of individual bacterial strain cultures was poured onto the solid medium. After solidification of top agar, 10 μl of bacteriophage lysate with the titer of at least 1×10⁶ PFU/ml was applied. The plates were left until the spot-applied phages were absorbed and then incubated for 18-24 hours at 37° C. After incubation, the sensitivity of the bacterial strains to the tested bacteriophages was determined. APEC strain was considered sensitive to the tested phage when the bacteria growth was inhibited in the spot application area. The results of specificity determination (host range) of selected bacteriophages against 86 different APEC strains are shown in Table 1.

TABLE 1
Specificity of bacteriophages towards APEC strains.

| | APEC [symbol] |
|---|---|
| [symbol] | [symbol] |

[symbol]

indicates data missing or illegible when filed

### Genetic Characteristics of Bacteriophages

Fifty bacteriophages were selected for a genetic analysis based on specificity results. The selected phages were subjected to differentiation with the obtained amplification (RAPD) or restriction (RFLP) profiles on the first stage of the genetic analysis. To that end, the phages' genomic DNA was isolated, using a modified method developed by Su et al. [Su M. T. et al., 1998]. The RAPD and RFLP analyses revealed that the tested group contained 47 different bacteriophage strains.

The results obtained in the initial genotypic differentiation enabled the further selection of the bacteriophages which DNA was sequenced using the Next Generation Sequencing (NGS) method on Illumina platform. The obtained results were submitted de novo (SPAdes 3.11.1) and manually processed (FA_TOOL; UŁ), and the obtained sequences were annotated in DNA Master. Next, bioinformatic analysis was carried out to determine the bacteriophage replication cycle. Seventeen of the forty-five sequenced bacteriophage strains were considered virulent because no genes responsible for the performance of a lysogenic cycle were found in the bioinformatic analyses.

### Analysis of Bacteriophages Specificity Towards Various APEC Serotypes

The next stage involved an in silico analysis of a collection of APEC strains to determine the strain serotypes based on the bacterial genomes' bioinformatic analysis. Then the specificity results for the isolated virulent bacteriophages were referred to different E. coli serotypes occurring most often in the APEC collection of Proteon Pharmaceuticals S.A., including but not limited to O2, O4, O8, O25, O50/O2, O78, O88, O117 and O161 serotypes, characterised as those that most often cause poultry infections.

The results of the studies allowed to compose BAFACOL preparation containing five bacteriophages (215Ecol030PP; 216Ecol046PP; 232Ecol030PP; 235Ecol030PP, and 236Ecol005PP) selected among virulent phages characterised by a broad and mutually complementary host range (Table 1). Table 2 summarises the specificity spectrum (expressed in % of sensitive strains among the tested group of the particular serotype strains) of phages included in BAFACOL preparation against different APEC serotypes that most often induce poultry infections. The composed preparation contains phages representing different and mutually complementary specificity profiles, which ensures its activity towards a wide range of APEC strains and reduces the probability of the bacteria becoming resistant to the preparation.

TABLE 2
BAFACOL preparation specificity towards different APEC serotypes.

| | APEC serotypes | | | | | | | | | |
|---|---|---|---|---|---|---|---|---|---|---|
| | (number of strains) | | | | | | | | | |
| | O2 | O4 | O8 | O25 | O50/O2 | O78 | O88 | O117 | O161 | Other |
| | (7) | (1) | (6) | (2) | (7) | (18) | (5) | (3) | (5) | (32) |
| BAFACOL | 86 | 100 | 33 | 100 | 100 | 100 | 100 | 100 | 100 | 56 |
| preparation | | | | | | | | | | |
| specificity | | | | | | | | | | |
| [%] | | | | | | | | | | |

### Comparison of the Specificity of BAFACOL Preparation and Phages Included in Its Composition.

Then, using the spot test method, the host range (specificity) was evaluated and compared for the developed BAFACOL preparation and individual bacteriophages included in its composition (Table 3). It was demonstrated that 78% of 86 tested APEC strains were sensitive to individual phages included in BAFACOL preparation, whereas their sensitivity to BAFACOL preparation amounted to 95%.

TABLE 3
Specificity of BAFACOL preparation and individual bacteriophages
included in its composition was evaluated using the spot test method.

| | APEC [symbol] |
|---|---|
| [symbol] | [symbol] |

[symbol]

indicates data missing or illegible when filed

### Example 2. Demonstrating a Synergistic Effect of the Phages Included in BAFACOL Preparation

In order to evaluate the synergistic action of the phages in BAFACOL preparation and to demonstrate the advantage of the developed preparation over other phage cocktails, the lytic activity of individual bacteriophages and their combination towards BAFACOL preparation was compared.

Into each well in a 96-well plate, 100 μl of 100-fold diluted 20-hour Escherichia coli_014PP2015 bacterial culture was added, followed by 10 μl of bacteriophage suspension in the amount of 2×10⁵ PFU/well for the tests of individual phages or 10 μl of bacteriophage suspension in the amount of 2×10⁵ PFU/well for tested cocktail, multiplied by the number of the cocktail ingredients. At the same time, positive control containing only the medium with the bacterial culture was prepared. Wells containing only the medium or phage lysate were the sterility and background controls. The absorbance of the tested samples was measured at the wavelength of λ=620 nm, at the microplate reader, at 20 minutes' intervals, during incubation for 1400 minutes at 37° C.

The numerical data obtained in the test were used for calculating the coefficient of synergism (S), which helps to determine the interaction of the phages used in the cocktail towards the tested bacteria strain:
S=1 neutral action
S>1 synergistic action
S<1 antagonistic action

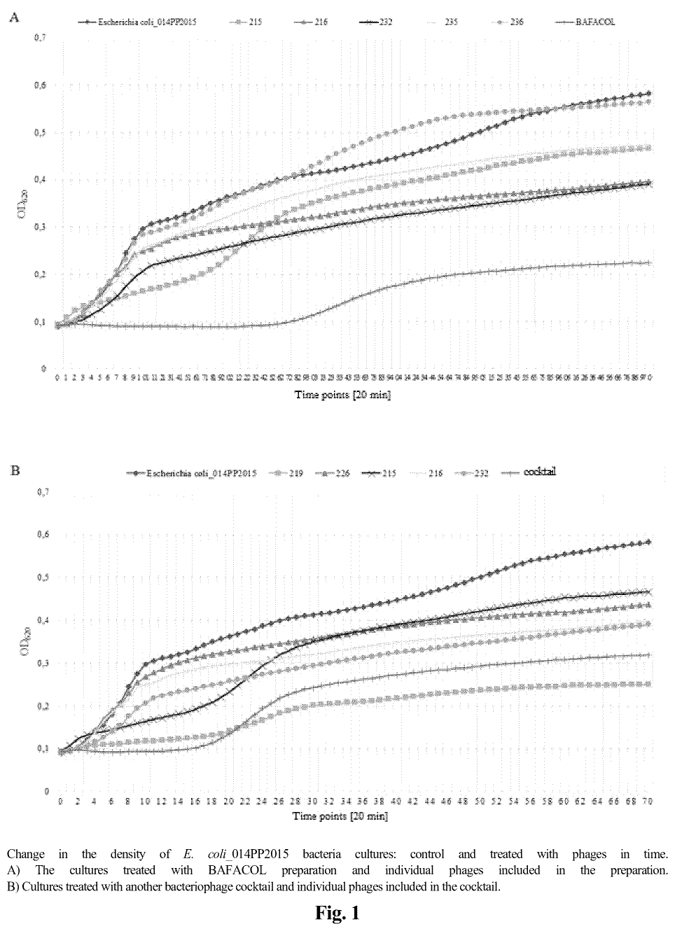

The first stage of coefficient S determination involved plotting the curves based on the obtained optical density values for the control and phage-treated bacterial cultures (FIG. 1).

Then the plotted curves were used to calculate the areas under the curves (P), using the formula [Xie Y. et al., 2018]:

$$P = {\sum }_{i = 1}^{70} \frac{O {D}_{i + 1} + O {D}_{i}}{2} .$$

On the following stage, the areas calculated for each tested system (P_{K} for the control culture and P_{B} for the phage-treated cultures) were standardised (N) by dividing the value of the area under the curve obtained for the bacteria growth control (P_{K}) by the value of the area under the curve obtained for individual test systems (P_{B}) (Tables 4 and 5). For the growth control, the P_{K} value was used instead of P_{B}, and the N value of 1 was obtained. The higher the N value obtained for the test system, the stronger the antibacterial effect of the tested bacteriophage or cocktail towards the applied bacterial strain was.

Coefficient S was determined as a ratio of the cocktail standardised value to the standardised value of the component with the strongest action.

TABLE 4
Coefficient S calculated for BAFACOL preparation.

| | | N value | |
|---|---|---|---|
| | Area | of the | |
| | under | bacteria | |
| | the | growth | |
| | curve | control | Coefficient S |
| | (P_{K}) | (P_{K}/P_{K}) | (S_{BAFACOL/232Ecol030PP}) |
| Bacteria | 28.90 | 1 | |
| growth control | | | |
| | Area | | |
| | under | | |
| | the | | |
| | curve | N value | |
| Bacteriophages | (P_{B}) | (P_{K}/P_{B}) | |
| 215Ecol030PP | 23.01 | 1.26 | |
| 216Ecol046PP | 22.02 | 1.31 | |
| 232Ecol030PP | 20.29 | 1.42 | |
| 235Ecol030PP | 25.46 | 1.13 | |
| 236Ecol005PP | 29.69 | 0.97 | |
| BAFACOL | 10.48 | 2.76 | 1.94 |

TABLE 5
Coefficient S calculated for the cocktail of 215Ecol030PP, 216Ecol046PP,
219Ecol002PP, 226Ecol053PP, and 232Ecol030PP phages.

| | Area under | N value of the | |
|---|---|---|---|
| | the curve | bacteria growth | Coefficient S |
| | (P_{K}) | control (P_{K}/P_{K}) | (S_{Cocktail/219Ecol002PP}) |
| Bacteria growth | 28.90 | 1 | |
| control | | | |
| | Area under the | N value | |
| Bacteriophages | curve (P_{B}) | (P_{K}/P_{B}) | |
| 215Ecol030PP | 23.01 | 1.26 | |
| 216Ecol046PP | 22.02 | 1.31 | |
| 219Ecol002PP | 13.33 | 2.17 | |
| 226Ecol053PP | 24.23 | 1.19 | |
| 232Ecol030PP | 20.29 | 1.42 | |
| Cocktail | 15.53 | 1.86 | 0.86 |

The result obtained for BAFACOL preparation suggests that bacteriophages in the cocktail, once used together, reveal a synergistic effect and inhibit the bacteria growth more effectively than each phage used separately (Table 4). In order to demonstrate that the result observed for BAFACOL preparation applied only to adequately selected bacteriophage compositions, an analogical test was carried out for another bacteriophage cocktail which included the following phages: 215Ecol030PP, 216Ecol046PP, 219Ecol002PP, 226Ecol053PP and 232Ecol030PP (Table 5). In this case, combining the bacteriophages into a cocktail has an antagonistic impact on the action of 219Ecol002PP phage which, when used separately, inhibited the growth of Escherichia coli_014PP2015 more effectively than the cocktail did.

The summary of the obtained results allowed to demonstrate that the method presented in the description of the invention enabled the development of a unique bacteriophage preparation—BAFACOL, which composition is highly active towards APEC strains sensitive to it and minimises the likelihood of the bacteria becoming resistant to the preparation.

### Example 3. Characteristics of BAFACOL Preparation Components

### Taxonomy of BAFACOL Preparation Bacteriophages

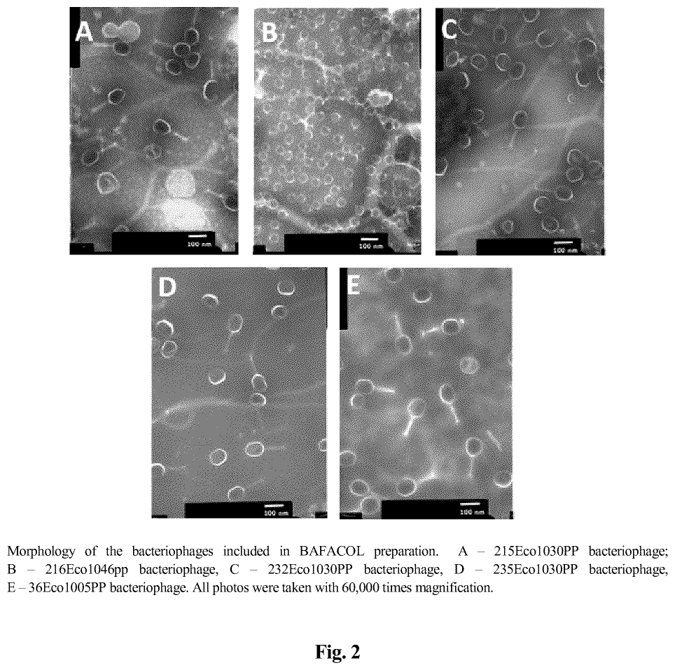

215Ecol030PP, 216Ecol046PP, 232Ecol030PP, 235Ecol030PP and 236Ecol005PP bacteriophages selected as the components of BAFACOL preparation were subjected to full genetic characteristics to examine their similarity to the reference phages and to carry out visualisation using a transmission electron microscope (Table 6, FIG. 2).

TABLE 6
Characteristics of bacteriophages included in BAFACOL preparation.

| | | Bacteriophage | | | | |
|---|---|---|---|---|---|---|
| Feature | | 215Ecol030PP | 232Ecol030PP | 235Ecol030PP | 236Ecol030PP | 216Ecol030PP |
| Host: E. coli | | 030PP2016 | 030PP2016 | 030PP2016 | 005PP2016 | 053PP2016 |
| Material | | Wastewater | Wastewater | Wastewater | Wastewater | Wastewater |
| | | Municipal and | Municipal and | Wastewater | Municipal and | Municipal and |
| | | Housing Services | Housing Services | Treatment Plant | Housing Services | Housing Services |
| | | Department in | Department in | in Łódź | Department in | Department in |
| | | Stryków | Stryków | | Stryków | Stryków |
| | | | | | | icozaedral capside |
| | | | | | | and a short non- |
| Morphology | | icozaedral capside and a long contractile tail | | | | contractile tail |
| Double-stranded DNA size [bp] | | 168,618 | 167,665 | 167,860 | 167,663 | 40,043 |
| Taxonomy | Order | Caudovirales | Caudovirales | Caudovirales | Caudovirales | Caudovirales |
| of the reference | Family | Myoviridae | Myoviridae | Myoviridae | Myoviridae | Podoviridae |
| strain | Subfamily | Tevenvirinae | Tevenvirinae | Tevenvirinae | Tevenvirinae | Autographivirinae |
| | Genus | Tequatrovirus | Tequatrovirus | Tequatrovirus | Tequatrovirus | Teseptimavirus |
| | | (T4 virus) | (T4 virus) | (T4 virus) | (T4 virus) | (T7virus) |
| | Species | Escherichia phage | Escherichia phage | Escherichia coli | Enterobacteria | Enterobacteria |
| | | YUEEL01 | vB_Ecom- | O157 typing | phage Kha5h | phage K1F |
| | | | fFiEco06 | phage 7 | | |

| | | | | | |
|---|---|---|---|---|---|
| Analysed phage genome coverage | 93 | 98 | 90 | 92 | 93 |
| compared to the reference genome | | | | | |

In order to take photos of the phages using a transmission electron microscope, the bacteriophages included in the preparation were centrifuged at 24,500×g for 3 hours and rinsed, and the obtained sediment was suspended in ammonium molybdate. A ten-fold dilution of each sample was then prepared, which was then applied to the formvar and carbon coated copper gird, and contrasted in the darkness with phosphotungstic acid. The photos of the bacteriophages were taken in JEOL 1010 TEM electron microscope in the Laboratory of Microscopic Imaging and Specialized Biological Techniques, Faculty of Biology and Environmental Protection, University of Łódź. The morphology of the bacteriophages is shown in FIG. 2.

### Testing Sensitivity of the Variants Resistant to Selected Bacteriophages

In order to check whether the phages used in BAFACOL preparation will exhibit lytic activity against the possible emerging variants of bacteria resistant to individual phages included in the preparation, a study was carried out to isolate variants of E. coli_030PP2016, E. coli_053PP2016 and E. coli_005PP2016 strains resistant to each of the phages included in BAFACOL preparation composition.

100 μl of phage with the titer of ca. 1×10⁹ PFU/ml and 100 μl of adequately diluted bacteria (host) culture were added to Eppendorf tubes. After 10 minutes of incubation at 37° C., 100 μl sample was taken and spread over the surface of a solid medium. The obtained individual bacteria colonies were used to start liquid cultures which were used to titer the tested phage with a double-layered plate method. The bacteria were considered resistant (resistant variant to specific bacterial strain) to the particular phage when no bacterial lawn lysis or plaques were observed on the bacterial lawn. The spot test was then applied to check if the other phages selected for the cocktail remained active towards the obtained resistant variants.

The E. coli_030PP2016 strain resistant variants were obtained only for 215Ecol030PP phage. The 232Ecol030PP and 235Ecol030PP phages did not induce resistant mutants among E. coli_030PP2016 bacteria. The variants resistant to 215Ecol030PP phage were still sensitive to 216Ecol046PP, 232Ecol030PP and 236Ecol005PP phages. For E. coli_053PP2016 strain, variants resistant to 216Ecol046PP phage were obtained. The variants were still sensitive to 232Ecol030PP, 235Ecol030PP and 236Ecol005PP phages. For E. coli_005PP2016 no mutants resistant to 236Ecol005PP phage were obtained.

Summing up, BAFACOL preparation's composition ensures the maintenance of the preparation lytic activity towards the variants of bacteria resistant to individual bacteriophages included in BAFACOL preparation.

### Frequency of Occurrence of Mutants Resistant to Individual Bacteriophages Included in BAFACOL Preparation and to BAFACOL Preparation

E. coli_030PP2016 and E. coli_053PP2016 strains were used in the tests, for which mutants resistant to individual phages from BAFACOL preparation were obtained. Among the phages included in the preparation, E. coli_030PP2016 strain demonstrates sensitivity to 215Ecol030PP, 232Ecol030PP and 235Ecol030PP bacteriophages, whereas E. coli_053PP2016 strain is sensitive to 215Ecol030PP and 216Ecol046PP phages. This fact was taken into consideration in the concentrations of the phages used for the assessment of the occurrence frequency of resistant mutants.

100 μl of BAFACOL preparation with the titer of 1×10⁸ PFU/ml or 215Ecol030PP, 232Ecol030PP, and 235Ecol030PP bacteriophages, with the titer of 6×10⁷ PFU/ml or 215Ecol030PP and 216Ecol046PP with the titer of 4×10⁷ were added to each Eppendorf test tube. Then 100 μl of 100-fold dilution of bacteria culture with the density of OD₆₀₀=0.5 (ca. 10⁶ CFU/ml) were added to the test tubes. Simultaneously, a bacteria control sample was prepared, containing 100 μl of the growth medium and 100 μl of the bacteria culture. The samples were incubated for 10 minutes at 37° C. The top agar containing 100 μl of BAFACOL preparation or 100 μl of individual bacteriophages was poured onto the plates with the growth medium. The top agar with 100 μl of the solution used to suspend the bacteriophages was poured onto the plates intended for inoculation of bacteria from the control sample. Then, the plates were streaked with 100 μl of individual samples. The plates were incubated for 24 hours at 37° C. After incubation, the number of grown colonies on individual plates was counted. The colonies grown on the plates with BAFACOL preparation or individual bacteriophages were inoculated on subsequent plates containing the tested bacteriophages to confirm the persistence of phage resistance in the bacterial cells. The plates were incubated for 24 hours at 37° C. The colonies were counted after the incubation, and the number of mutants resistant to BAFACOL preparation and individual bacteriophages was determined. The frequency of occurrence of mutants resistant to BAFACOL preparation and individual tested bacteriophages was calculated taking into account the number of bacteria obtained in the control sample and the number of the mutants resistant to the tested phages (Table 7).

TABLE 7
Frequency of occurrence of mutants resistant to individual bacteriophages
and BAFACOL preparation.

| | Number of | Frequency of resistant |
|---|---|---|
| | resistant mutants | mutants formation |
| | Escherichia coli_030PP2016 (4 × 10⁶ CFU/ml) | |
| BAFACOL | 0 | 0 |
| 215Ecol030PP | 11 | 3.69 × 10⁻⁴ |
| 232Ecol030PP | 0 | 0 |
| 235Ecol030PP | 0 | 0 |
| | Escherichia coli_053PP2016 (5.8 × 10⁶ CFU/ml) | |
| BAFACOL | 0 | 0 |
| 215Ecol030PP | 62 | 9.35 × 10⁻³ |
| 216Ecol046PP | 119 | 4.87 × 10⁻³ |

The study showed that BAFACOL preparation prevents the bacteria from developing resistance to phages, illustrated by the example of E. coli_053PP2016 strain. Considering the sensitivity of the applied method, it can be concluded that BAFACOL preparation reduced the frequency of the emergence of phage resistant mutants by at least 10,000 times. Moreover, the differences in the number of induced mutants resistant to 215Ecol030PP bacteriophage were observed between the tested bacterial strains, but no resistance of the strains to BAFACOL preparation containing the abovementioned bacteriophage was demonstrated.

### Testing the Bacteriophages Lytic Activity

The bacteriophages included in BAFACOL preparation were subjected to a lytic activity test. To that end, 100 μl of 100-fold diluted ca. 20-hour bacterial culture was added to four wells in a 96-well plate. 100 μl of the growth medium (positive control) was added to two of them, and 100 μl of the particular bacteriophage (test sample) in the amount of 2×10⁶ PFU/well was added to the other two wells. Additionally, only the growth medium was applied into two wells (200 μl) and only the bacteriophage lysate (200 μl) into the other two wells, which were the negative controls. Then the absorbance (OD₆₂₀) of the tested samples was measured with a microplate reader at 20 minutes' intervals, during incubation for 300 minutes at 37° C. Based on the results of the optical density (OD) measurement, the degree of the bacterial strain growth inhibition by the tested bacteriophage was determined, assuming that:
the difference between the positive control OD and the test sample OD>0.1 means strong inhibition of the bacteria growth by the tested phage;
the difference between the positive control OD and the test sample OD between 0.1 and 0.05 means weak inhibition of the bacteria growth by the tested phage;
the difference between the positive control OD and the test sample OD<0.05 means no influence of the tested phage on the bacteria growth.

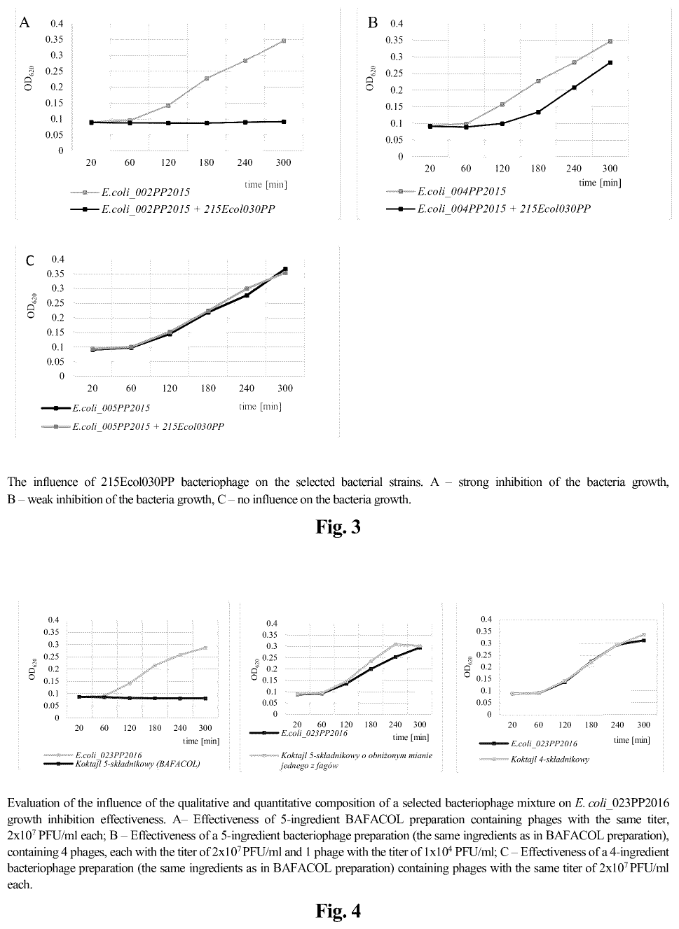

FIG. 3 shows the results obtained for three bacteria strains in a lytic activity test of 215Ecol030PP bacteriophage, as an example. The selected bacterial strains are characterised by a diversified sensitivity to the tested phage and represent all cases analysed in the lytic activity test.

The lytic activity test of the phages included in BAFACOL preparation revealed a significant influence of the bacteriophages on the growth of the tested APEC strains (Table 8). According to the lytic activity test results, the phages strongly inhibited the growth of 58 tested strains (67.4%), slightly inhibited the growth of 5 tested strains (5.8%), and showed no influence on the growth of 23 tested strains (26.7%).

TABLE 8
Lytic activity of each phage included in BAFACOL preparation.

| | APEC [symbol] |
|---|---|
| [symbol] | [symbol] |

[symbol]

indicates data missing or illegible when filed

### Example 4. Testing the Influence of the Quantity and the Number of Phage in the Selected Bacteriophage Composition on Its Lytic Activity

In order to evaluate the influence of the qualitative and quantitative composition of the selected bacteriophage formulation, the effectiveness was tested of BAFACOL preparation (215Ecol030PP; 216Ecol046PP; 232Ecol030PP; 235Ecol030PP, 236Ecol005PP), where the bacteriophage titers were equal and amounted to 2×10⁷ PFU/ml (FIG. 4A); of a 5-ingredient cocktail (215Ecol030PP; 216Ecol046PP; 232Ecol030PP; 235Ecol030PP, 236Ecol005PP) in which one phage titer was 1×10⁴ PFU/ml, whereas the titer of the other four phages amounted to 2×10⁷ PFU/ml (FIG. 4B); and of a 4-ingredient cocktail (215Ecol030PP; 216Ecol046PP; 232Ecol030PP; 235Ecol030PP) in which the bacteriophage titers were equal and amounted to 2×10⁷ PFU/ml (FIG. 4C). The effectiveness of the phages was evaluated with the lytic activity test.

By comparing the effectiveness of BAFACOL preparation, developed as part of the invention, with a preparation with a lower number of phages or containing one of the phages at a lower titer, it was demonstrated that a lack or reduction in the content of phage in the preparation has a negative influence on the effectiveness of E. coli_023PP2016 strain growth inhibition.

### Example 5. Testing the Drug Sensitivity of Selected APEC Strains Compared with Sensitivity to BAFACOL Preparation

BAFACOL preparation with the total titer of 1×10⁸ PFU/ml was used in the tests. The preparation did not reveal any presence of microorganisms, which was confirmed in microbiological purity tests.

The drug sensitivity of APEC strains was tested with a disk diffusion method, according to CLSI recommendations (Clinical Laboratory Standards Institute; 2008 document: Performance standards for antimicrobial disk and dilution susceptibility tests for bacteria isolated from animals; approved standard, 3rd Ed. CLSI document M31-A3. Clinical and Laboratory Standards Institute, Wayne, PA.). The disk diffusion method used disks soaked with 10 selected drugs (Table 9).

All selected APEC strains were resistant to ampicillin, nalidixic acid, streptomycin and sulfonamide The highest drug resistance was revealed for E. coli_130PP2017 strain, which was sensitive only to apramycin, among all tested antibiotics and chemotherapeutics. The other tested APEC strains were resistant to 6-8 test antimicrobial drugs. All the strains revealed sensitivity to BAFACOL preparation.

TABLE 9
Comparison of the sensitivity of selected APEC strains to drugs and phages

| | | | Chloram- | Gen- | Kana- | Nalidixic | Strepto- | Sulfo- | Tetra- | | |
|---|---|---|---|---|---|---|---|---|---|---|---|
| E. coli | Ampicillin | Apramycin | phenicol | tamicin | mycin | acid | mycin | namide | cycline | Trimethoprim | |
| strain | 10 μg | 15 μg | 30 μg | 10 μg | 30 μg | 30 μg | 10 μg | 30 μg | 30 μg | 5 μg | BAFACOL |
| 130PP2017 | R | I | R | R | R | R | R | R | R | R | + |
| 156PP2017 | R | I | R | S | R | R | R | R | R | R | + |
| 023PP2016 | R | I | S | S | R | R | R | R | R | R | + |
| 035PP2016 | R | I | R | S | S | R | R | R | R | R | + |
| 054PP2016 | R | I | R | S | S | R | R | R | R | R | + |
| 081PP2016 | R | I | R | S | I | R | R | R | R | R | + |
| 016PP2015 | R | I | S | S | S | R | R | R | R | R | + |
| 021PP2016 | R | I | R | S | S | R | R | R | R | S | + |
| 037PP2016 | R | I | R | S | I | R | R | R | S | R | + |
| 077PP2016 | R | I | I | S | S | R | R | R | R | R | + |
| 376PP2016 | R | I | I | R | S | S | R | R | R | S | + |
| 377PP2016 | R | I | R | I | S | R | R | R | R | S | + |

### Example 6. Testing temperature Stability of BAFACOL Preparation

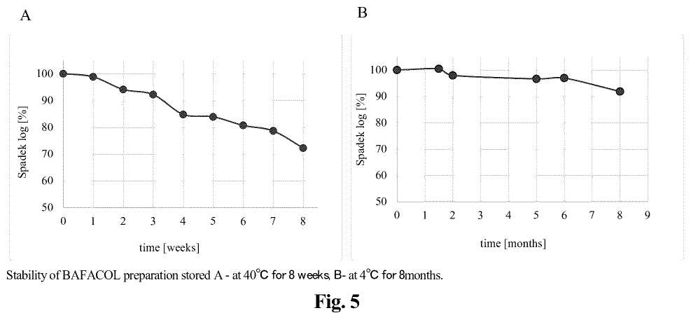

The storage stability study of BAFACOL preparation with a total titer of 1×10⁸ PFU/ml, was carried out at 4° C. for 8 months and at 40° C. for 8 weeks (FIG. 5). The phage titer was identified with a double-layered plate method.

The storage stability studies results reveal that BAFACOL preparation is characterised by high storage stability and maintains the bacteriophage titer at 92% (expressed in % log₁₀) for 8 months at 4° C. and 72% for 8 weeks at 40° C., which is essential for the product life.

### Example 7. Assessment of the Effectiveness of Using BAFACOL Preparation in Drinking Water to Protect Poultry from APEC Strains

BAFACOL preparation with the total titer of 1×10⁸ PFU/ml was used in the tests. The preparation did not reveal any presence of microorganisms, which was confirmed in microbiological purity tests. The study was carried out in cooperation with AgriSearch Hungary Ltd.

The research material consisted of 384 one-day broiler chickens, randomly divided into 3 equal groups (T1, T2 and T3), kept in 24 units of 16 birds each. There were 8 units in each group. Any broilers with injuries or ill-health were excluded from the test. The birds were fed with complete feed mixtures from Szindbad Ltd., 2100 Gödöllő, Mikszáth Kálmán u. 10, Hungary, which composition complied with the commercial feeding requirements for broilers. The complete feed mixtures did not contain antibiotics or growth stimulators. The conditions in which the broilers were kept complied with the requirements of the breeding material manufacturer. During the experiment, BAFACOL preparation was administered to the broilers, and they were infected with E. coli APEC bacteria (O78:H4 serotype) according to the following pattern:
the broilers in T1 group (negative control group) were not infected with APEC strain and were not given BAFACOL preparation,
the broilers in T2 group (positive control group) were infected with APEC strain in the dose of 10⁶ CFU/bird on day 14 of the experiment, and were not given BAFACOL preparation,
the broilers in T3 group were infected with APEC strain in the dose of 10⁶ CFU/bird on day 14 of the experiment and were given BAFACOL preparation as a potable water additive, in the dose of 10⁶ PFU/bird starting from the first day of the experiment, every other day.

The following parameters were monitored during the entire experiment period (day 0-35 of the experiment):
mortality,
zootechnical parameters:
   BW—mean body weight,
   ADFI—average daily feed intake,
   ADG—average daily weight gain,
   ADWI—average daily water intake,
   FCR—feed conversion ratio,
   EPEF—European production efficiency factor.

The results of the evaluated parameters are summarised in Table 1.

TABLE 10
The influence of BAFACOL preparation administered as a potable water
additive on the tested parameters in the broiler on days 0-35 of the
experiment (Number of birds = 384, mean values ± standard deviation,
abc statistical significance at p ≤ 0.05).

| | Test group | | |
|---|---|---|---|
| Tested parameter | T1 | T2 | T3 |
| Mortality [%] | 1.6 ± 2.9ª | 26.6 ± 9.3^{b} | 5.5 ± 5.2ª |
| Body weight [g/bird] | 1,937 ± 28ª | 1,642 ± 71^{b} | 1,751 ± 120^{c} |
| Average daily weight gain | 54.0 ± 0.6ª | 41.8 ± 1.5^{b} | 48.2 ± 3.0^{c} |
| [g/bird/day] | | | |
| Feed intake [g/bird/day] | 88.4 ± 2.0ª | 81.5 ± 3.9^{b} | 88.9 ± 2.7ª |
| Water intake [mL/bird/day] | 162.6 ± 4.5ª | 124.7 ± 8.6^{b} | 155.8 ± 7.8ª |
| FCR [kg/kg] | 1.64 ± 0.03ª | 1.95 ± 0.12^{b} | 1.85 ± 0.09^{b} |
| EPEF | 325 ± 11ª | 158 ± 24^{b} | 247 ± 7^{c} |

*^{abc}Different superscripts stand for statistically significant differences; statistical analysis was carried out in the columns.

Based on the obtained results it was demonstrated that the mortality of broilers in the group receiving BAFACOL preparation as a drinking water additive (T3) decreased nearly five times compared to the positive control group (T2). Moreover, it was demonstrated that the average daily body weight gain, the broilers' body weight, feed consumption and water consumption were significantly higher in the group receiving BAFACOL preparations as a drinking water additive (T3) as compared to the positive control group (T2) but slightly lower than in the negative control group (T1). The EPEF factor was higher in the group receiving BAFACOL as a drinking water additive (T3), while the FCR stayed at a high level in this group but was still lower than in the positive control group (T2).

The summary of the test of the effectiveness of using BAFACOL bacteriophage preparation in drinking water to protect poultry from APEC strains demonstrated that BAFACOL preparation:
1. effectively prevents the mortality of birds exposed to infection with APEC strains.
2. significantly improves the zootechnical parameters of birds exposed to infection with APEC strains.
3. is effective in preventing colibacillosis in broilers.
4. is a preparation promising for use in the prevention of colibacillosis in broilers.

### Example 8. Assessment of the Effectiveness of Using BAFACOL Bacteriophage Preparation Spray to Protect Poultry from APEC Strains

BAFACOL preparation with the total titer of 1×10⁸ PFU/ml was used in the tests. The preparation did not reveal any presence of microorganisms, which was confirmed in microbiological purity tests. The study was carried out in cooperation with AgriSearch Hungary Ltd.

The research material consisted of 384 one-day broiler chickens, randomly divided into 3 equal groups (T1, T2 and T3), kept in 24 units of 16 birds each. There were 8 units in each group. Any broilers with injuries or ill-health were excluded from the test. The birds were fed with complete feed mixtures from Szindbád Ltd., 2100 Gödöllő, Mikszáth Kálmán u. 10, Hungary, which composition complied with the commercial feeding requirements for broilers. The complete feed mixtures did not contain antibiotics or growth stimulators. The conditions in which the broilers were kept complied with the requirements of the breeding material manufacturer. During the experiment, BAFACOL preparation was administered to the broilers, and they were infected with E. coli APEC bacteria (O78:H4 serotype) according to the following pattern:
the broilers in T1 group (negative control group) were not infected with APEC strain and were not given BAFACOL preparation,
the broilers in T2 group (positive control group) were infected with APEC strain in the dose of 10⁶ CFU/bird on day 14 of the experiment, and were not given BAFACOL preparation,
the broilers in T3 group were infected with APEC strain in the dose of 10⁶ CFU/bird on day 14 of the experiment, whereas BAFACOL preparation was used in the form of a spray in every unit, in the dose of 8×10⁶ PFU/m² before the broilers were placed, and then every other day throughout the experiment.

The following parameters were monitored during the entire experiment period (day 0-35 of the experiment):
mortality
zootechnical parameters:
   mean body weight,
   ADFI—average daily feed intake,
   ADG—average daily weight gain,
   ADWI—average daily water intake,
   FCR—feed conversion ratio,
   EPEF—European production efficiency factor.

The results of the evaluated parameters are summarised in Table 11.

TABLE 11
The influence of BAFACOL preparation administered as a spray on
the tested parameters in the broilers on days 0-35 of the experiment
(Number of birds = 384, mean values ± standard deviation, abc
statistical significance at p ≤ 0.05).

| | Test group | | |
|---|---|---|---|
| Tested parameter | T1 | T2 | T3 |
| Mortality [%] | 1.6 ± 2.9ª | 26.6 ± 9.3^{b} | 7.0 ± 4.0ª |
| Body weight [g/bird] | 1,937 ± 28ª | 1,642 ± 71^{b} | 1,736 ± 91^{bc} |
| Average daily weight gain | 54.0 ± 0.6ª | 41.8 ± 1.5^{b} | 47.6 ± 2.0^{c} |
| [g/bird/day] | | | |
| Feed intake [g/bird/day] | 88.4 ± 2.0ª | 81.5 ± 3.9^{b} | 84.9 ± 3.0^{ab} |
| Water intake [mL/bird/day] | 162.6 ± 4.5ª | 124.7 ± 8.6^{b} | 155.7 ± 4.6ª |
| FCR [kg/kg] | 1.64 ± 0.03^{a} | 1.95 ± 0.12^{b} | 1.78 ± 0.07^{c} |
| EPEF | 325 ± 11^{a} | 158 ± 24^{b} | 248 ± 10^{c} |

*^{abc}Different superscripts stand for statistically significant differences; statistical analysis was carried out in the columns.

Based on the obtained results, it was demonstrated that the mortality of broilers in the group receiving BAFACOL preparation spray (T3) decreased nearly four times as compared to the positive control group (T2). Moreover, it was demonstrated that the mean daily gain in the body weight, the broilers' body weight, feed consumption and water consumption were significantly higher in the group receiving BAFACOL preparation spray (T3) as compared to the positive control group (T2) but slightly lower than in the negative control group (T1). The EPEF factor was higher in the group receiving BAFACOL as a spray (T3), while the FCR stayed at a high level in this group but was still lower than in the positive control group (T2).

The summary of the study of the effectiveness of using BAFACOL bacteriophage preparation spray to protect poultry from APEC strains demonstrated that BAFACOL preparation:
1. effectively prevents the mortality of birds exposed to infection with APEC strains.
2. significantly improves the zootechnical parameters of birds exposed to infection with APEC strains.
3. minimises the likelihood of APEC strains becoming resistant.
4. is effective in preventing colibacillosis in broilers.
5. is a preparation promising for use in the prevention of colibacillosis in broilers.

### REFERENCES

Short-term outlook for EU agricultural markets in 2018 and 2019, Summer 2019.
Mellata M. Human and avian extraintestinal pathogenic Escherichia coli: Infections, zoonotic risks and antibiotics resistance trends. Foodborne Pathogens Dis. 2013, 10(11): 916-931.
Dho-Moulin M, Fairbrother J M. Avian pathogenic Escherichia coli (APEC). Vet. Res. 1999, 30: 299-316.
Furfaro L L, Payne M S, Chang B J. Bacteriophage Therapy: Clinical Trials and Regulatory Hurdles. Front Cell Infect Microbiol. 2018, 8: 376.
Huff W E, Huff G R, Rath N C, Donoghue A M. Method of administration affects the ability of bacteriophage to prevent colibacillosis in 1-day-old broiler chickens. Poult Sci. 2013, 92(4): 930-934.
Huff W E, Huff G R, Rath N C, Balog J M, Donoghue A M. Alternatives to antibiotics: utilisation of bacteriophage to treat colibacillosis and prevent foodborne pathogens. Poult Sci. 2005, 84(4): 655-659. 10.1093/ps/84.4.655.
Oliveira A, Sillankorva S, Quinta R, Henriques A, Sereno R, Azeredo J. Isolation and characterisation of bacteriophages for avian pathogenic E. coli strains. J Appl Microbiol. 2009, 106(6): 1919-1927.
Huff W E, Huff G R, Rath N C, Donoghue A M. Evaluation of the influence of bacteriophage titer on the treatment of colibacillosis in broiler chickens. Poult Sci. 2006, 85(8): 1373-1377.
Jamalludeen N, She Y M, Lingohr E J, Griffiths M. Isolation and characterisation of virulent bacteriophages against Escherichia coli serogroups O1, O2 and O78. Poult Sci. 2009, 88: 1694-1702.
Lau G L, Sieo C C, Tan W S, Hair-Bejo M, Jalila A, Ho Y W. Efficacy of a bacteriophage isolated from chickens as a therapeutic agent for colibacillosis in broiler chickens. Poult Sci. 2010, 89(12): 2589-2596.
Huff W E, Huff G R, Rath N C, Balog J M, and Donoghue A M. Prevention of Escherichia coli infection in broiler chickens with a bacteriophage aerosol spray. Poult. Sci. 2002a, 81: 1486-1491.
Huff W E, Huff G R, Rath N C, Balog J M, Donoghue A M. Prevention of Escherichia coli infection in broiler chickens with a bacteriophage aerosol spray. Poult Sci. 2002b, 81(10): 1486-1491.
Dunnington E A, Siegel P B, Gross W B. Escherichia coli challenge in chickens selected for high or low antibody response and differing in haplotypes at the major histocompatibility complex. Avian Dis. 1991, 35(4): 937-940.
Gomis S M, Watts T, Riddell C, Potter A A, Allan B J. Experimental reproduction of Escherichia coli cellulitis and septicemia in broiler chickens. Avian Dis. 1997, 41(1): 234-240.
Naghizadeh M, Karimi Torshizi M A, Rahimi S, Dalgaard T S. Synergistic effect of phage therapy using a cocktail rather than a single phage in the control of severe colibacillosis in quails. Poult Sci. 2019, 98(2): 653-663.
Huff W E, Huff G R, Rath N C, Balog J M, Donoghue A M. Bacteriophage treatment of a severe Escherichia coli respiratory infection in broiler chickens. Avian Dis. 2003a, 47: 1399-1405.
Huff W E, Huff G R, Rath N C, Balog J M, Donoghue A M. Evaluation of aerosol spray and intramuscular injection of bacteriophage to treat an Escherichia coli respiratory infection. Poult Sci. 2003b, 82: 1108-1112.
El-Gohary F A, Huff W E, Huff G R, Rath N C, Zhou Z Y, Donoghue A M. Environmental augmentation with bacteriophage prevents colibacillosis in broiler chickens. Poult Sci. 2014, 93(11): 2788-2792.
WO2019051603A1
KR101275263B1
EP3133151A1
US20160083695A1
WO2015160164A1
KR101299179B1
KR101830900B1
KR101871338B1
KR20120111535A
Barrow P, Lovell M, Berchieri Air. Use of lytic bacteriophage for control of experimental Escherichia coli septicemia and meningitis in chickens and calves. Clin Diagn Lab Immunol. 1998, 5: 294-298.
Huff W E, Huff G R, Rath N C, Donoghue A M. Immune interference of bacteriophage efficacy when treating colibacillosis in poultry. Poult Sci. 2010, 89(5): 895-900.
Patent application No. P. 431942
Su M T, Tyamagondlu V V, Bodmer R. Large- and small-scale preparation of bacteriophage lambda lysate and DNA. BioTechniques 1998, 25(1): 44-6.
Xie Y, Wahab L, Gill J J. Development and Validation of a Microtiter Plate-Based Assay for Determination of Bacteriophage Host Range and Virulence. Viruses 2018, 10(4): 189.

1. A process for preparing a synergistic phage composition characterised in that:
a) at least two different phage strains are obtained, specific towards the same bacteria strain,
b) bactericidal activity of each phage strain separately and their mixture is determined towards this bacteria strain,
c) bactericidal activity determined for each phage strain used separately is compared with the bactericidal activity determined for their mixture, and
d) if the bactericidal activity of a mixture is discovered to be higher than the bactericidal activity of each phage separately, the phages are considered to form a synergistic composition.

2. A process according to claim 1, characterised in that on stage b) in order to determine the bactericidal activity of phages, bacteria strain is cultivated in the presence of the studied phages and a bacteria strain is cultivated without the phages, the growth curves are plotted for each culture, and the areas under the curves are determined, whereby the ratio of the area under the growth curve determined for the culture without the phages to the area under the growth curve determined for the bacteria culture incubated with the studied phages is regarded as the measure of the studied phages bactericidal activity, and preferably the value of the coefficient N standing for the ratio is determined.

3. A process according to claim 1, characterised in that on stage b) the growth curves are determined, by measuring the absorbance time changes at the wavelength λ=620 nm of the samples collected from the tested bacteria strain culture.

4. A process according to claim 1, characterised in that on stage d) the value of coefficient S is determined, being the ratio of the mixture bactericidal activity to the bactericidal activity of the phage strain with the strongest action in the mixture, whereby the mixture is regarded as a synergistic phage composition if the value of coefficient S is greater than 1.

5. A process according to claim 1, characterised in that on stage a) in order to obtain a phage strain specific towards the selected bacteria strain:
a collection of bacteriophage strains is obtained, containing a bacteriophage strain specific towards the selected bacteria strain,
a selected bacteria strain is cultivated on a sterile culture medium,
culture samples of individual bacterial strains suspended in the agar are poured onto a solid culture medium, after the top agar has solidified, the suspension of the tested bacteriophage strain with the titer of at least 1×10⁶ PFU/m1 is spotted and incubated at 37° C. for at least 18 h,
after the incubation, the clear zones are checked in the area where the given bacteriophage strain suspension was applied, which means the inhibition of the bacteria growth; For a bacteriophage strain specific against a selected bacteria strain, clear zones are observed within the top agar,
genetic analysis of selected phages is carried out with the RAPD or RFLP method and sequencing with the NGS method, which enables the exclusion of the phages which perform a lysogenic cycle, and possibly
the identified bacteriophage strain specific for bacterial strains of different serotypes is propagated.

6. A process according to claim 1, characterised in that APEC strain is the selected bacteria strain.

7. A synergistic composition of phages specific towards APEC bacteria, containing at least two bacteriophage strains deposited in the Polish Collection of Microorganisms at deposit numbers: F/00137 (215Ecol030PP strain), F/00138 (216Ecol046PP strain), F/00139 (232Ecol030PP strain), F/00140 (235Ecol030PP strain), and F/00141 (236Ecol005PP strain).

8. A composition according to claim 7 for use in the treatment or prevention of livestock infections, including but not limited to poultry, with APEC strains.

9. A composition for use according to claim 8 characterised in that it is to be administered to the threatened animals as a drinking water additive or spray.

10. A composition for use according to claim 8 characterised in that it ensures a significant reduction in the mortality of poultry infected with APEC strain.

11. A composition for use according to claim 8 characterised in that it ensures a significant improvement in the zootechnical parameters (BW, ADFI, ADG, ADWI, FCR and EPEF) in poultry infected with APEC strain.

12. A composition for use according to claim 8 characterised in that it remains stable for at least 8 months at 4° C. and at least 8 weeks at 40° C.

13. A bacteriophage strain suitable for preventing or combatting infections with APEC strains, selected from a group consisting of: F/00137 (215Ecol030PP strain), F/00138 (216Ecol046PP strain), F/00139 (232Ecol030PP strain), F/00140 (235Ecol030PP strain), and F/00141 (236Ecol005PP strain).
